(19) United States
(12) Patent Application Publication
(10) Pub. No.: US 2012/0089184 A1
(43) Pub. Date: Apr. 12, 2012

(54) DEVICE FOR TIGHTLY GRIPPING ON THE SPINOUS PROCESS OF THE SPINE

(76) Inventor: CHUNG-CHUN YEH, Taipei City (TW)

(21) Appl. No.: 13/171,453

(22) Filed: Jun. 29, 2011

(30) Foreign Application Priority Data
Oct. 8, 2010 (TW) 099134648

(51) Int. Cl. A61B 17/70

(52) U.S. Cl. 606/248

(57) ABSTRACT

Disclosed is a device for tightly gripping on the spinous process, installed between two spinous processes, with a horizontal hole through the main body, and two arc-shaped side plates connecting onto two ends of the main body, each plate having a through hole, and fastening on the spinous process, and a centrally placed item passing through a horizontal hole of the main body thereby connecting the main body and the two plates, and firmly fastened by a single or plurality of fastening items on one or two sides of the centrally placed item. Furthermore, the centrally placed item having at least one fixed hole section, and when the fastening items move toward the main body and tighten, the two plates change shape, putting pressure on the upper and lower gripping section of the two plates, this upper and lower gripping section therefore more firmly gripping on the spinous process.

### BACKGROUND OF THE INVENTION

1. Field of the Invention

The present invention relates to a device for tightly gripping on the spinous process of the spine, in particular, to a device set between the adjacent spinous processes of the vertebrae in the body, having a centrally placed item and one or more fastening items, respectively putting pressure on the two arc-shaped side plates, and further firmly gripping on the spinous processes of the vertebrae.

2. Description of Related Art

In traditional spinal illnesses, excluding injuries resulting from broken bones in an external injury, a quite large ratio is pathological changes or atrophy caused by the invertebral disc, resulting in narrowing of the gap between the vertebrae of the spine, the joints between the vertebrae and the neural foramina (foramen intervertebral) between the joints also relatively narrowing, thereby putting pressure on the nerve roots of the spinal cord, resulting in paralysis or pain.

Currently, in general treatment of the above mentioned problem, besides implanting a pedicle screw and a rod to pull open the gap between the vertebrae, or implanting an artificial disk cage or spacer to fill the gap between the vertebrae so as to heighten the pad between the vertebrae of the spine and thus pull open the gap between the neural foramina, there isn't an improved surgical procedure to make surgery simpler, allow for easier placement, and a safer and more effective implant of an artificial spine object, to solve this problem.

Therefore a way is desired to solve the above mentioned problem, the key being how to enlarge the neural foramen between the vertebrae so as to relieve the pressure on the nerve root of the spinal cord, but requiring a simple and effective surgical procedure and yet maintaining the last big opportunity for a surgical procedure on the spine, and whereby these upper and lower joints in adjacent vertebrae can be completely fixed and stable. More specifically, these upper and lower spinous processes of the vertebrae and the device grow and merge to become one body thereby increasing the stability after installation in the surgical procedure, moreover treating the instability between the adjacent upper and lower centra of the vertebrae in a way to so as not to cause uncomfortable nerve pain. Therefore this invention (A device for tightly gripping on the spinous process of the spine) was developed whereby the upper and lower joints of adjacent vertebrae, according to a special device to open the spinous processes, are completely fixed and stable, and pressure is effectively relieved on the nerve root and additionally, a last opportunity to implant a pedicle screw is maintained.

### SUMMARY OF THE INVENTION

A device to solve the above mentioned deficiency is to implant the device for tightly gripping on the spinous process of the spine of the present invention between a spinous process of an upper vertebra and a spinous process of a lower vertebra.

One purpose of the invention is to provide a device for tightly gripping on the spinous process of the spine, designed with a horizontal hole in the main body, and connecting two arc-shaped side plates to two ends of the main body, and the arc-shaped side plates each having a through hole, fixed to the spinous processes of the vertebrae through the gripping section of the arc-shaped side plates, then inserting a centrally placed item horizontally into the main body to connect the main body and the arc-shaped side plates, finally one or more fastening items are fixed on one end or two ends of the centrally placed item.

characterized in that: the centrally placed item has at least one fixed hole section or stopper, which is fixed on the fixed hole section or a tightening end by at least one fastening item, and moves in the direction of the main body and tightens, the resulting force results in the arc-shaped side plates changing shape, putting pressure on a plurality of V-shapes on the upper and lower gripping sections to pierce and tightly fasten the spinous processes of the upper and lower vertebrae.

Another purpose of this invention is to provide a device for tightly gripping on the spinous processes of upper and lower vertebrae on the spine, which has a main body, two arc-shaped side plates, a centrally placed item and one or more fastening items, directly in contact with the upper and lower vertebrae and firmly fastening for fixation.

An additional purpose of the invention is to provide a device for tightly gripping on the spinous process of the spine. Two arc-shaped side plates respectively set a through hole connecting two ends of the main body, the arc-shaped side plates respectively having upper and lower gripping sections, fixed on the spinous processes of the upper and lower vertebrae, a centrally placed item passing through the two arc-shaped side plates and the main body, and finally a fastening item fixed on one end or two ends of the centrally placed item, causing the upper and lower gripping sections to pierce and tightly grip on the spinous processes of the upper and lower vertebrae.

One more additional purpose of the invention is to provide a device for tightly gripping on the spinous process of the spine, wherein the centrally placed item has two ends, setting a fixed hole section with a plurality of sequential layers or a flange head as a stopper on one end. The arc-shaped side plates have different thickness, setting a number of convex ribs and changing shape as “S” under pressure. Every two of the plurality of V-shapes of the upper and lower gripping sections are disposed relative and non-linear to each other, and the plurality of V-shapes each has various lengths. The upper and lower gripping sections of the arc-shaped side plates have one or more kerf so as to form multiple parallel plates.

Therefore, a device of the invention for tightly gripping on the spinous process of the spine and is set between a spinous process of an upper vertebra and a spinous process of a lower vertebra. The main body has upper and lower surfaces in contact with the spinous processes of the upper and lower vertebrae, and a horizontal hole set through the main body. Two arc-shaped side plates respectively set on the through hole, connecting two ends of the main body, the arc-shaped side plates respectively having upper and lower gripping sections, fixing on the spinous processes of the upper and lower vertebrae. A centrally placed item passes through the horizontal hole to connect the main body and the arc-shaped side plates, and at least one fastening item is fixed on two ends of the centrally placed item. Since the two ends of the centrally placed item have a fixed hole section with a plurality of sequential layers, and at least one fastening item able to encircle, plug into or screw into, a layer, hole or thread of the fixed hole section, when the one or more fastening items encircle, plug into or screw into the layer, hole or thread of the two ends of the centrally placed item, the arc-shaped side plates are pushed to contact the spinous processes of the upper and lower vertebrae, the fastening item is facing the main body, and is closer to the subsequent layer, hole or thread, the one or more fastening items encircle, plug into or screw into for tightening, resulting in the arc-shaped side plates changing shape and, putting pressure on a plurality of V-shapes on the upper and lower gripping sections to pierce and tightly grip on the spinous processes of the upper and lower vertebrae.

In this embodiment, the fixed hole section of the centrally placed item has a columnar section on each end with a plurality of sequential layers set on each columnar end section, the plurality of sequential layers being ring-shaped steps sequentially facing the main body, and the fastening item being a flexible fixed gasket so as to affix onto the plurality of ring-shaped steps.

In this embodiment, the fixed hole section of the centrally placed item has a columnar section on each end with a plurality of sequential layers set on each columnar end section, the plurality of sequential layers having a plurality of holes sequentially facing the main body, and the fastening item being a plug or screw which can plug into or screw into the plurality of holes.

In this embodiment, the fixed hole section of the centrally placed item has a columnar section on each end with a plurality of sequential layers set on each columnar end section, the plurality of sequential layers having a threaded section facing the main body, and the fastening item being a nut so as to screw onto the threaded section.

In this embodiment, when pressure is exerted on the arc-shaped side plates by the fastening item, the upper and lower gripping sections of the arc-shaped side plates will generate a transformation with a relative distance, and the middle section thereof will generate a relative transformation close to “S” shape will transform to form an approximate “S” shape.

In this embodiment, the upper and lower gripping sections of the arc-shaped side plates have a plurality of V-shapes, each two opposing points of the plurality of V-shapes are disposed relative but non-linear to each other, and the plurality of V-shapes each have differing lengths.

In this embodiment, the upper and lower gripping sections of the arc-shaped side plates have one or more kerf so as to form multiple parallel plates.

In this embodiment, the arc-shaped side plates have a number of convex ribs or a number of sunken grooves to increase the downward pressure and fixing stability, and prevent revolutions generated from the arc-shaped side plates.

In this embodiment, the thicknesses of the upper and lower gripping sections are designed thicker than the middle section thereof. Therefore, these thinner portions in the middle section of the arc-shaped side plates will generate downward pressures so as to result in the arc-shaped side plates firmly fastening on the spinous processes of the upper and lower vertebrae, the fixing stability is increased by a number of vertical, horizontal or slanted convex ribs set on the middle section.

In this embodiment, the main body is a saddle-type item, having a bottom plate connecting two vertical plates. The bottom plate and the vertical plates create an accommodating space, where synthetic bone can be implanted so as to merge the spinous processes of the upper and lower vertebrae as a unit. The vertical plate sets the horizontal hole to provide for the penetration of the centrally placed item or the bottom of the main body sets similar to the tapered, which can be implanted between the upper and lower spinous processes of the vertebrae.

Additionally, the invention further provides a device for tightly gripping on the spinous process of the spine, which is set between a spinous process of an upper vertebra and a spinous process of a lower vertebra, a main body having upper and lower surfaces, in contact with the spinous processes of the upper and lower vertebrae, and a horizontal hole through the main body; two arc-shaped side plates each having a through hole, connecting two ends of the main body, the arc-shaped side plates respectively having an upper and lower gripping section, fixed on the spinous processes of the upper and lower vertebrae; a centrally placed item having one end and the other end, the centrally placed item passing through the horizontal hole of the main body to connect the main body and the arc-shaped side plates; and finally at least one fastening item fixed on the outside end of the centrally placed item, moreover, a flange head stopper set on one end of the centrally placed item for tightly against the boundary of the through hole in one of the arc-shaped side plates, a tightening end set on the other end of the centrally placed item, the centrally placed item passing through the through hole of the arc-shaped side plates and the horizontal hole of the main body, the tightening end of the other end of the centrally placed item passing the through hole of the other one of the arc-shaped side plates, the fastening item fixed on the tightening end of the centrally placed item, when the fastening item moves in the direction of the main body and tightens, the flange head stopper of the centrally placed item contacts the fastening item, the resulting force results in the arc-shaped side plates changing shape, putting pressure on a plurality of V-shapes on the upper and lower gripping sections to pierce and tightly grip on the spinous processes of the upper and lower vertebrae.

In this embodiment, the stopper of the centrally placed item is a flange head, which fits tightly against the boundary of the through hole of the arc-shaped side plates.

In this embodiment, the tightening end of the centrally placed item is a threaded section and the fastening item is a nut, so as to screw tightly and fasten on the threaded section of the centrally placed item.

In this embodiment, the nut further has a screw hole tying in with a locking screw, which can lock the nut firmly on the centrally placed item.

In this embodiment, the tightening end of the centrally placed item is a ring-shaped step and the fastening item is a flexible fixed gasket so as to affix onto the ring-shaped steps, the flexible fixed gasket further able to move in the direction of the main body so as to affix firmly to the next stage of the plurality of ring-shaped steps.

The words “up, down, front and back” mentioned in the device for tightly gripping on the spinous process of the spine, which relatively contrast with the spine of a patient who lies on the operating table in the prone position for undergoing surgery, distinguishing “upper” as the head and “lower” as the feet. The normal visual direction is the front, and the opposite is the rear. The top and the bottom are the designated portion, which is judged by upwards or downwards coordinate of the relative average values, and indicated by the drawings and labels.

### BRIEF DESCRIPTION OF THE DRAWINGS

The invention, as well as its many advantages, may be further understood by the following detailed description and drawings in which:

FIG. 1a and FIG. 1b are the exploded schematic diagrams of the preferred embodiment showing the device for tightly gripping on the spinous process of the spine of the invention.

FIG. 1c is the schematic diagram showing the different thicknesses among the front, the middle and the rear portion of two side plates of the device for tightly gripping on the spinous process of the spine.

FIG. 1d is the schematic diagram showing a number of vertical, horizontal or diagonal convex ribs set in the middle section of two side plates of the device for tightly gripping on the spinous process of the spine.

FIG. 1e is the schematic diagram showing a number of convex ribs or a number of sunken grooves set in two side plates of the device for tightly gripping on the spinous process of the spine.

FIG. 1f is the schematic diagram showing the bottom of the main body set similar to the tapered of the device for tightly gripping on the spinous process of the spine.

FIG. 2 is the schematic diagram showing the altitude difference and a plurality of V-shapes disposed relative and non-linear to each other of the device for tightly gripping on the spinous process of the spine.

FIG. 3a and FIG. 3b are the exploded schematic diagrams of another preferred embodiment showing the device for tightly gripping on the spinous process of the spine of the present invention.

FIG. 4a, FIG. 4b and FIG. 5 are the schematic diagrams showing the combination and force direction of the device for tightly gripping on the spinous process of the spine set between the spinous processes of the upper and lower vertebrae.

### DETAILED DESCRIPTION OF THE INVENTION

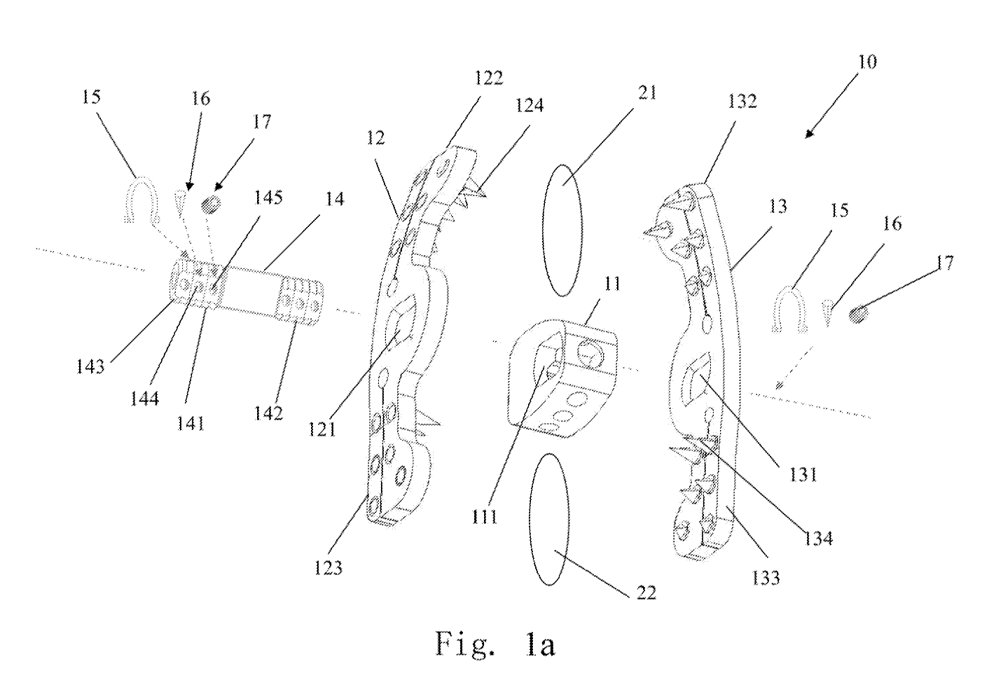

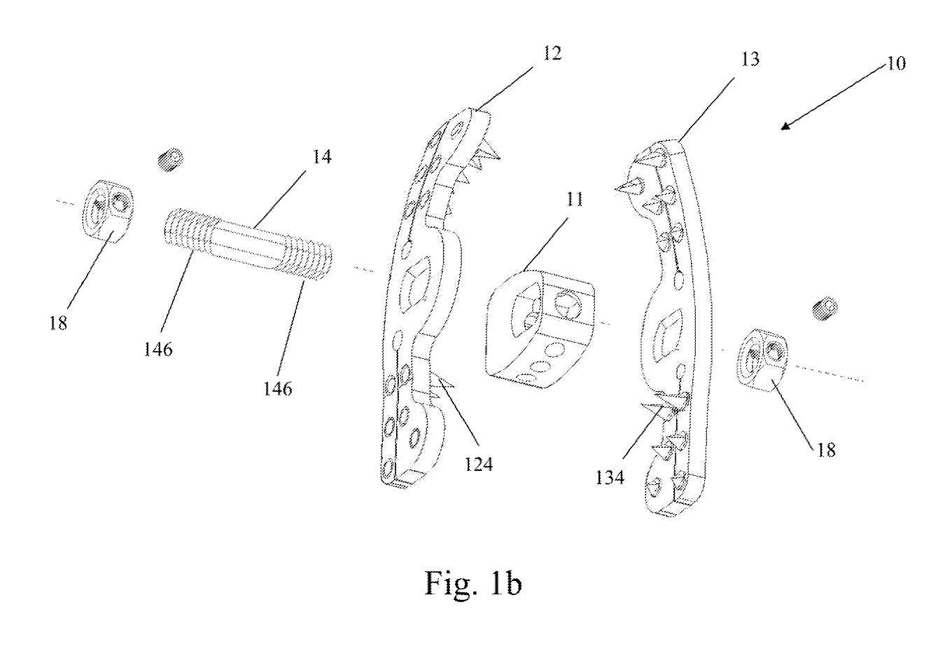

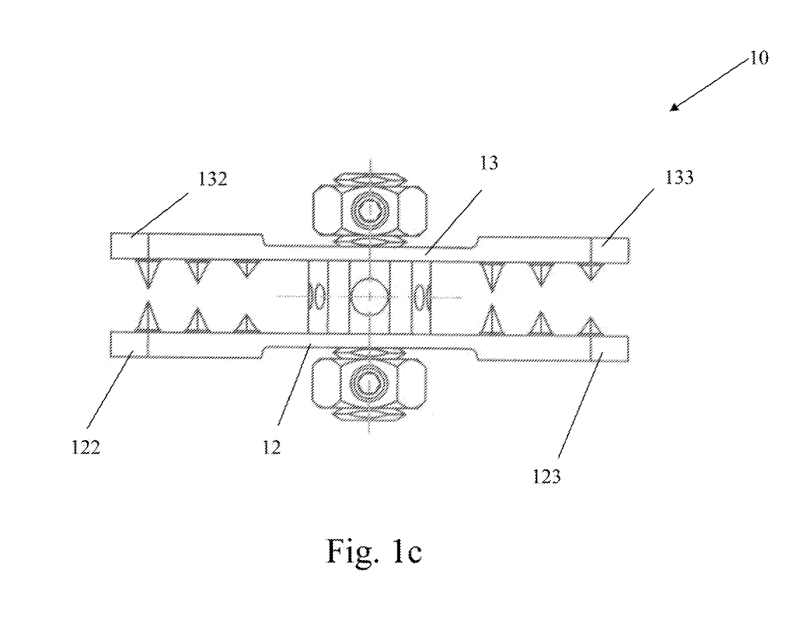

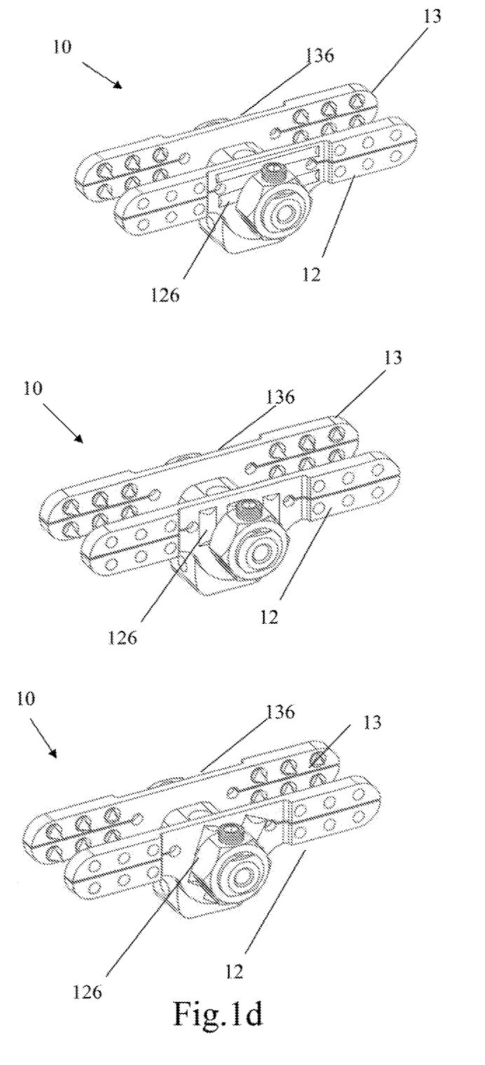

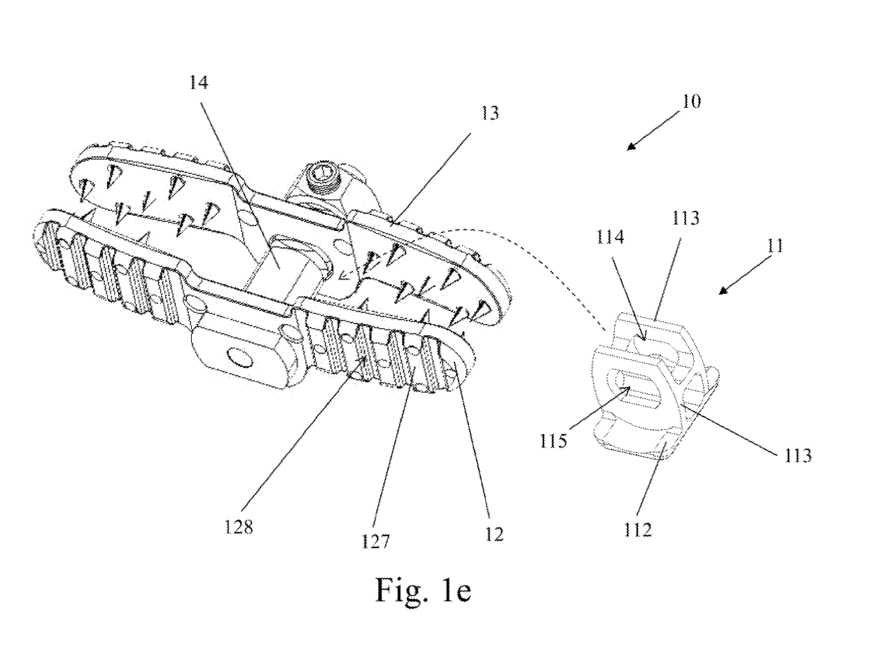

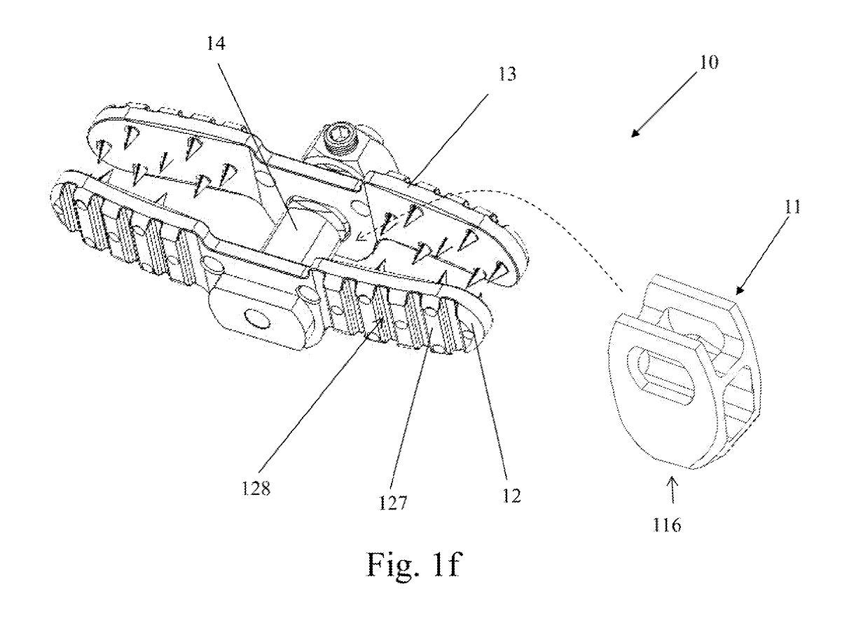

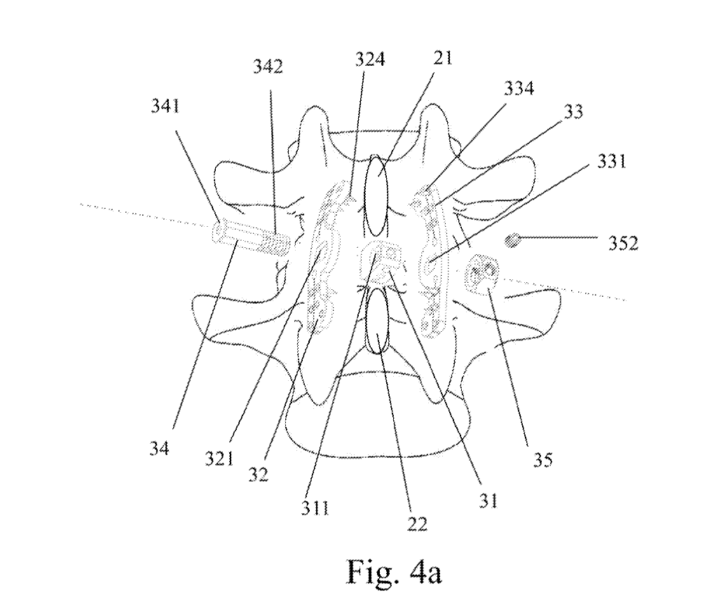

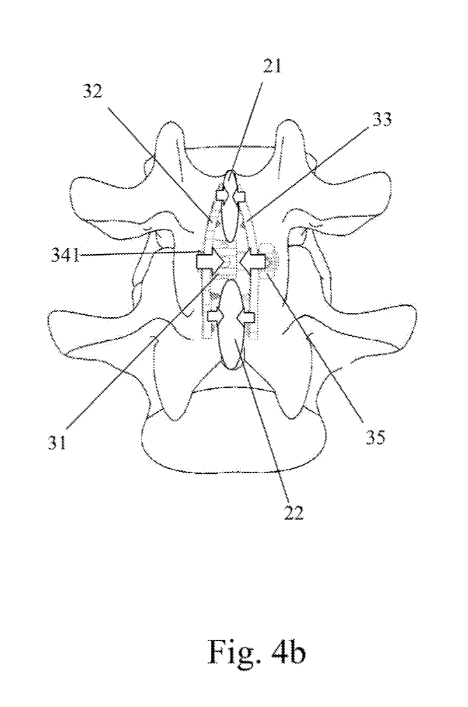

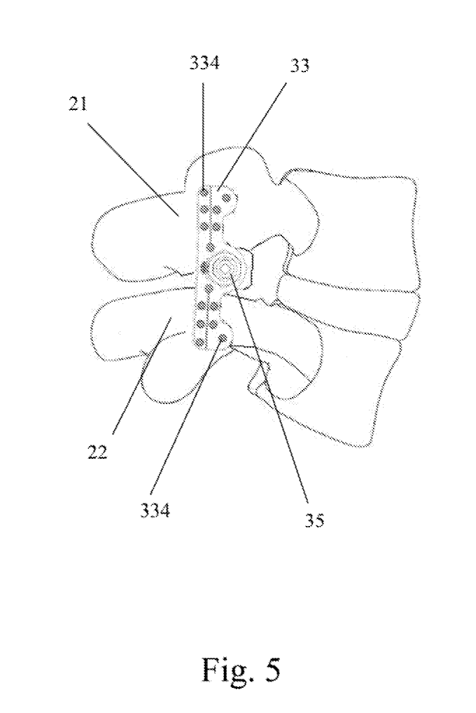

Please refer to FIG. 1a and FIG. 1b, which is a device for tightly gripping on the spinous process of the spine 10, including a main body 11, two arc-shaped side plates 12 and 13, a centrally placed item 14 and a variety of styles of multiple fastening items 15, 16 and 17; the device 10 is set between a spinous process of an upper vertebra 21 and a spinous process of a lower vertebra 22 (as shown in FIG. 4a).

The upper and lower surfaces of the main body 11 are in contact with the spinous processes of the upper and lower vertebrae 21 and 22, and a horizontal hole 111 set through the main body; the arc-shaped side plates 12 and 13 respectively set a through hole 121 and 131, connecting two ends of the main body 11, the arc-shaped side plates 12 and 13 setting an upper and lower gripping sections 122, 123, 132 and 133 respectively, fixing on the spinous processes of the upper and lower vertebrae 21 and 22; the centrally placed item 14 inserted through the horizontal hole 111 of the main body 11 to connect the arc-shaped side plates and the main body 11, and the fastening items 15, 16 and 17 fixed on the two ends 141 and 142 of the centrally placed item through various methods.

Wherein, two ends 141 and 142 of the centrally placed item set a plurality of sequential fixed hole section/each have a section with a plurality of sequential fixed holes, which is a columnar section similar to stacked layers, forming a plurality of sequential ring-shaped steps 143 thereon in the direction of the main body 11, at least one fastening item selected from a flexible fixed gasket so as to affix around at least one of the plurality of ring-shaped steps 143.

Additionally, the fixed hole section of the two ends 141 and 142 of the centrally placed item having a plurality of sequential holes 144 or threaded holes 145 facing the main body 11, the fastening items 15, 16 and 17 can be a plug 16 which can be plugged into the hole 144, or a screw 17, which can be screwed into the threaded hole 145 for fixation.

In FIG. 1b, a/the plurality of sequential fixed holes at the two ends of the centrally placed item 14 sequentially have a plurality of ring-shaped steps 143 thereon facing the main body 11, the at least one fastening item selected from a flexible fixed gasket so as to affix around at least one of the plurality of ring-shaped steps 143.

The fastening items 15, 16, 17 and 18 can affix around, plug into or screw into a layered step 143 or hole 144 and 145 or thread 146, putting pressure on the arc-shaped side plates 12 and 13 so as to be in contact with the spinous processes 21 and 22 of the upper and lower vertebrae.

Furthermore, the fastening items 15, 16, 17 and 18 move towards the main body 11 and closer to the subsequent layer 143, hole 144 and 145 or thread 16, when affixing around, plugging into or screwing into for tightening, which results in the arc-shaped side plates 12 and 13 changing shape, putting pressure on a plurality of V-shapes 124 and 134 on the upper and lower gripping sections 122, 123, 132 and 133 to pierce and tightly grip on the spinous processes 21 and 22 of the upper and lower vertebrae.

In FIG. 1c, the thickness of the upper and lower gripping sections 122, 123, 132 and 133 of the arc-shaped side plates 12 and 13 is thicker than the middle section thereof. Therefore, these thinner portions in the middle section of the arc-shaped side plates 12 and 13 will generate downward pressures so as to result in the arc-shaped side plates changing shape, thus putting pressure on a plurality of V-shapes on the upper and lower gripping sections thereby to pierce and tightly grip on the spinous processes of the upper and lower vertebrae 21 and 22.

In FIG. 1d, the arc-shaped side plates 12 and 13 have a number of vertical, horizontal or slanted convex ribs 126 and 136 to increase the downward pressure and the fixing stability and prevent revolutions generated from the arc-shaped side plates 12 and 13.

Moreover, in FIG. 1e, the arc-shaped side plates 12 and 13 have a number of convex ribs 127 and a number of sunken grooves 128 to increase the downward pressure and the fixing stability and prevent revolutions generated from the arc-shaped side plates 12 and 13. The main body 11 is further designed having a saddle shape, having a bottom plate 112 connecting the two vertical plates 113. The bottom plate 112 and the vertical plates 113 create an accommodating space 113, where synthetic bone can be implanted so as to merge the spinous processes of the upper and lower vertebrae 21 and 22 to be one unit. The vertical plate 113 sets the horizontal hole 115 to provide for the penetration of the centrally placed item 14.

In FIG. 1f, the bottom of the main body 11 is set similar to V-shaped 116, so that the main body 11 can be conveniently implanted between the spinous processes of the upper and lower vertebrae 21 and 22.

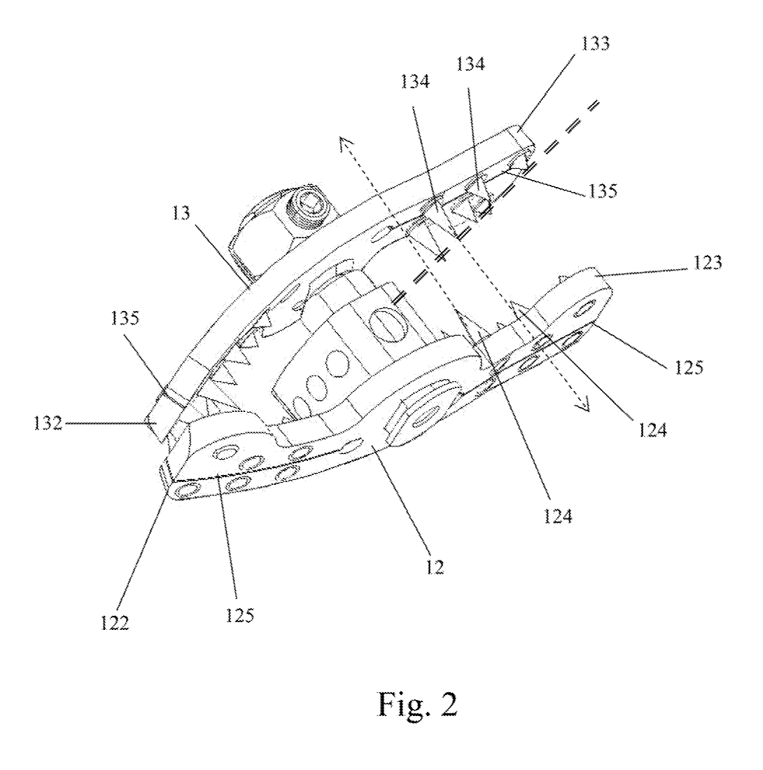

Please refer to FIG. 2, the upper gripping sections 122 and 132 and lower gripping sections 123 and 133 of the arc-shaped side plates 12 and 13 each have a plurality of V-shapes 124 and 134. Each two opposing points of the plurality of V-shapes are disposed relative and non-linear to each other as the arrow in the dotted line shows in the figure. The plurality of the V-shapes 124 and 134 of the upper and lower gripping sections 122 and 132 each have different lengths as the double dotted line shows in the figure.

The upper and lower gripping sections 122 and 132 of the arc-shaped side plates 12 and 13 having one or more kerf so as to make the upper and lower gripping section of the arc-shaped side plates 12 and 13 form the multiple parallel plates and to automatically adjust the side pieces of the affixture separately according to the different positions.

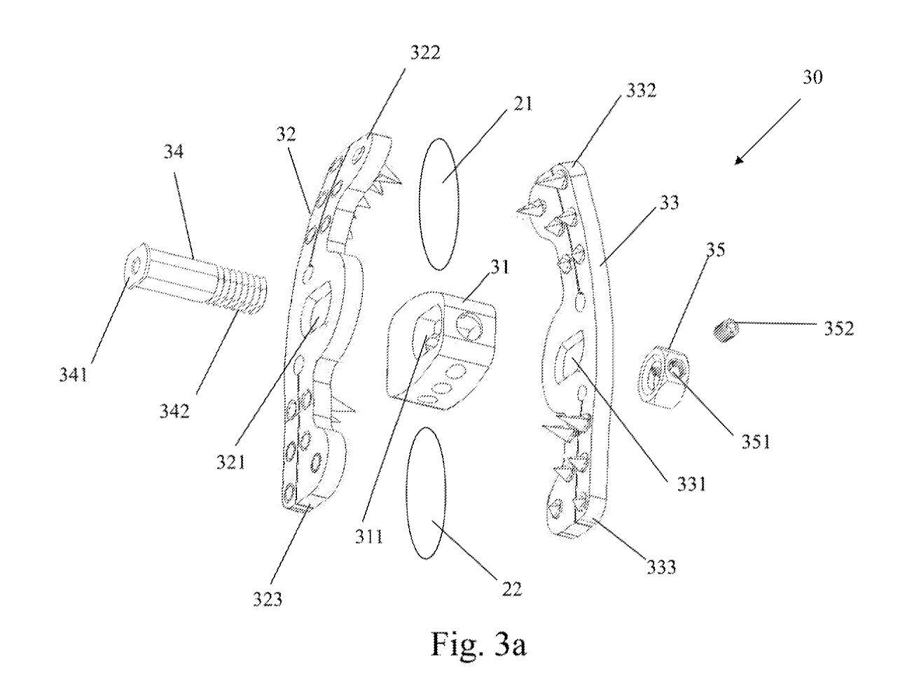

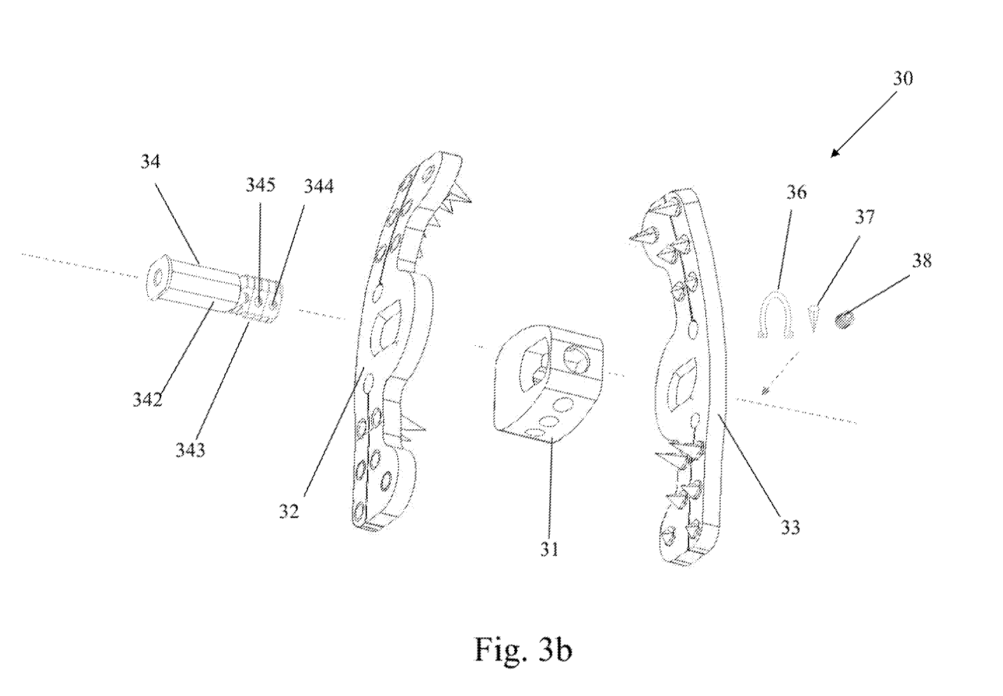

Please refer to FIG. 3a and FIG. 3b, the invention provides another device 30 for tightly gripping on the spinous process of the spine, which is also installed between a spinous process of an upper vertebra 21 and a spinous process of a lower vertebra 22, which includes a main body 31, two arc-shaped side plates 32 and 33, a centrally placed item 34 and the plurality of fastening items 35, 36, 37 and 38 having different styles.

The upper and lower surfaces of the main body 31 are in contact with the spinous processes of the upper and lower vertebrae 21 and 22, and a horizontal hole 311 set through the main body.

The two arc-shaped side plates 32 and 33 each have a through hole 321 and 331, connecting two ends of the main body 31, the arc-shaped side plates 32 and 33 having upper and lower gripping sections 322, 323, 332 and 333 respectively, which can affix on the spinous processes of the upper and lower vertebrae 21 and 22; when the centrally placed item 34 passes through the horizontal hole 311 of the main body 31 to connect the main body 31 and the arc-shaped side plates 32 and 33, which is fixed on the other end of the centrally placed item 34 by the fastening item 35.

Wherein, a stopper 341 is set on one end of the centrally placed item 34, a tightening end 342 is set on the other end of the centrally placed item. The stopper 341 is a flange head which fits tightly against the boundary of the through holes 321 and 331. When the fastening item 35 is a nut, the nut can be screwed tightly on the tightening end 342 of the centrally placed item 34.

Additionally the nut 35 has a locking screw 352, which screws into the screw hole 351 so as to lock the nut 35 firmly on the centrally placed item 34.

In FIG. 3b, the tightening end 342 of the centrally placed item 34 can be designed as ring-shaped steps 343 and the fastening item is then a flexible fixed gasket, which can affix around the ring-shaped steps 343 of the centrally placed item 34. The flexible gasket 36 which is affixed, further moves in the direction of the main body 31 so as to affix firmly to the next stage of the ring-shaped steps 343.

In addition, the tightening end 342 of the centrally placed item 34 sequentially has a plurality of holes 344 or threaded holes 345 facing the main body 31, the fastening item 37 and 38 being a plug 37 or screw 38 so as to plug into or screw into the plurality of holes 344 or threaded holes 345.

Please refer to FIG. 4a, FIG. 4b and FIG. 5, the centrally placed item 34 is inserted through the through hole 321 of the arc-shaped side plate 32, and passes through the horizontal hole 311 of the main body 31 to let the tightening end 342 of the centrally placed item 34 pass through the through hole 331 of the other arc-shaped side plate 33, then the tightening end 342 of the centrally placed item 34 is fastened by the fastening item 35. When the fastening item 35 moves towards the main body 31 for fixation, the stopper 341 of the centrally placed item 34 will be in contact with the fastening item 35 so that the resulting force causes the arc-shaped side plates 32 and 33 to change and look similar in shape to an S, thus putting pressure on a plurality of V-shapes 324 and 334 on the upper and lower gripping sections 322, 323, 332 and 333 and causing them to pierce and tightly grip on the spinous processes 21 and 22 of the upper and lower vertebrae.

Many changes and modifications in the above described embodiment of the invention can, of course, be carried out without departing from the scope thereof. Accordingly, to promote the progress in science and the useful arts, the invention is disclosed and is intended to be limited only by the scope of the appended claims.

What is claimed is:

1. A device for tightly gripping on a spinous process of a spine set between a spinous process of an upper vertebra and a spinous process of a lower vertebra, comprising:
a main body having upper and lower surfaces being in contact with the spinous processes of the upper and lower vertebrae, and a horizontal hole set through the main body;
two arc-shaped side plates respectively set at a through hole, connecting two ends of the main body, the arc-shaped side plates having an upper and lower gripping section respectively, affixing on the spinous processes of the upper and lower vertebrae;
a centrally placed item passing through the horizontal hole to connect the main body and the arc-shaped side plates; and
at least one fastening item affixing on two ends of the centrally placed item;
wherein the two ends of the centrally placed item have a fixed hole section with a plurality of sequential layers, the at least one fastening item being able to affix around, plug into or screw into a layer, hole or thread of the fixed hole section, when the at least one fastening item is affixed around, plugs into or screws into a layer, hole or thread of the two ends of the centrally placed item, thereby pushing the arc-shaped side plates to be in contact with the spinous processes of the upper and lower vertebrae, the fastening item facing the main body, and closer to the subsequent layer, hole or thread, the at least one fastening item affixing around, plugging into or screwing into, so as to result in the arc-shaped side plates changing shape and putting pressure on a plurality of V-shapes on the upper and lower gripping sections so as to tightly pierce and grip the spinous processes of the upper and lower vertebrae.

2. The device for tightly gripping on the spinous process of the spine of claim 1, wherein the fixed hole section having the plurality of sequential layers on the two ends of the centrally placed item, the plurality of sequential layers set on a columnar section and having a plurality of ring-shaped steps facing the main body thereon, the at least one fastening item being a flexible gasket so as to affix around the plurality of ring-shaped steps.

3. The device for tightly gripping on the spinous process of the spine of claim 1, wherein the fixed hole section has a plurality of sequential layers at the two ends of the centrally placed item, the plurality of sequential layers being set on a columnar section and having a plurality of ring-shaped steps facing the main body, and at least one fastening item being a plug or screw so as to plug into or screw into the plurality of holes or threaded holes.

4. The device for tightly gripping on the spinous process of the spine of claim 1, wherein the fixed hole section has a plurality of sequential layers at the two ends of the centrally placed item, the plurality of sequential layers set on a columnar section and having a plurality of ring-shaped steps facing the main body, and at least one fastening item being a nut so as to screw onto the threaded section.

5. The device for tightly gripping on the spinous process of the spine of claim 1, wherein each two opposing points of the plurality of V-shapes are disposed relative and non-linear to each other, the plurality of V-shapes each having varying lengths, the upper and lower gripping sections of the arc-shaped side plates having one or more kerf so as to form multiple parallel plates.

6. The device for tightly gripping on the spinous process of the spine of claim 1, wherein the arc-shaped side plates have a number of convex ribs and a number of sunken grooves.

7. The device for tightly gripping on the spinous process of the spine of claim 1, wherein the main body is a saddle-shaped item, having a bottom plate connected to two vertical plates, creating an accommodating space, the horizontal hole set through the vertical plate for providing the centrally placed item to penetrate and connect.

8. A device for tightly gripping on a spinous process of a spine set between a spinous process of an upper vertebra and a spinous process of a lower vertebra, comprising:
a main body having upper and lower surfaces, being in contact with the spinous processes of the upper and lower vertebrae, and a horizontal hole set through the main body;
two arc-shaped side plates each having a through hole, connecting two ends of the main body, the arc-shaped side plates having an upper and lower gripping section respectively, and affixing on the spinous processes of the upper and lower vertebrae;
a centrally placed item having one end and the other end, the centrally placed item inserted through the horizontal hole of the main body to connect the main body and the arc-shaped side plates; and
a fastening item affixing on the other end of the centrally placed item;
wherein a flange head stopper is set on one end of the centrally placed item for fitting tightly against the boundary of the through hole in one of the arc-shaped side plates, a tightening end set on the other end of the centrally placed item, the centrally placed item passing through the through hole of the arc-shaped side plates and the horizontal hole of the main body, the tightening end of the other end of the centrally placed item passing out the through hole of the other one of the arc-shaped side plates, the fastening item is affixed on the tightening end of the centrally placed item, when the fastening item moves in the direction of the main body and tightens, the flange head stopper of the centrally placed item is in contact with the fastening item, the resulting force causing the arc-shaped side plates to change shape, thereby putting pressure on a plurality of V-shapes on the upper and lower gripping sections to tightly pierce and grip the spinous processes of the upper and lower vertebrae.

9. The device for tightly gripping on the spinous process of the spine of claim 8, wherein the tightening end of the centrally placed item is a threaded section and the fastening item is a nut, so as to screw tightly and be fastened on the threaded section of the centrally placed item, the nut further having a locking screw which screws into the screw hole, so as to lock the nut firmly on the centrally placed item.

10. The device for tightly gripping on the spinous process of the spine of claim 8, wherein the tightening end of the centrally placed item is a plurality of ring-shaped steps and the fastening item is a flexible gasket so as to affix around the plurality of ring-shaped steps, the flexible gasket further moving in the direction of the main body to affix firmly to the subsequent stage of the plurality of ring-shaped steps.
